(19) United States
(12) Patent Application Publication
(10) Pub. No.: US 2024/0285852 A1
(43) Pub. Date: Aug. 29, 2024

(54) SYSTEMS AND METHODS FOR DETECTING FLUID INFILTRATION

(71) Applicant: Welch Allyn, Inc., Skaneateles Falls, NY (US)

(72) Inventors: Gene J. Wolfe, Skaneateles Falls, IN (US); WonKyung McSweeney, Skaneateles Falls, NY (US); David Kellner, Skaneateles Falls, NY (US)

(73) Assignee: Welch Allyn, Inc, Skaneateles Falls, NY (US)

(21) Appl. No.: 18/588,540

(22) Filed: Feb. 27, 2024

Related U.S. Application Data
(60) Provisional application No. 63/487,374, filed on Feb. 28, 2023.

(51) Int. Cl. A61M 5/168; A61M 5/142

(52) U.S. Cl. CPC A61M 5/16831; A61M 5/142; A61M 2005/14208

(57) ABSTRACT

Devices, systems, and methods for detecting a fluid infiltration event. A system includes a fluid delivery cannula having a distal end and a proximal end, a fluid source fluidly coupled to the proximal end of the fluid delivery cannula, and an optical sensor optically coupled to an interior of the fluid delivery cannula. The optical sensor is disposed between the distal end and the proximal end of the fluid delivery cannula and configured to generate an electronic signal based on one or more characteristics of light sensed in the interior of the fluid delivery cannula when fluid from the fluid source is passed therethrough. The system further includes a controller configured to receive the electronic signal from the optical sensor and determine a fluid infiltration event based on the electronic signal.

### BACKGROUND

### Field

The present disclosure relates to systems and methods for detecting fluid infiltration.

### Technical Background

Fluids are injected into subjects for a variety of reasons, such as to deliver medication or nutrients to a subject. Injections can involve the use of a needle to pierce a subject's tissue in order to inject the fluid. If the needle is improperly placed into the tissue (e.g., the needle misses a vein entirely or pierces completely through the vein and out the other side of the vein), fluid infiltration may occur.

### SUMMARY

In one embodiment, a system for detecting fluid infiltration includes a fluid delivery cannula which includes a distal end and a proximal end, a fluid source fluidly coupled to the proximal end of the fluid delivery cannula, an optical sensor optically coupled to an interior of the fluid delivery cannula, wherein the optical sensor is disposed between the distal end and the proximal end of the fluid delivery cannula and configured to generate an electronic signal based on one or more characteristics of light sensed in the interior of the fluid delivery cannula when fluid from the fluid source is passed therethrough, and a controller configured to receive the electronic signal from the optical sensor and determine a fluid infiltration event based on the electronic signal.

In another embodiment, fluid infiltration detection device includes an optical sensor optically coupled to a fluid delivery cannula delivering fluid from a fluid source therethrough and positioned at a location remote from a distal end of the fluid delivery cannula, the optical sensor configured to generate an electronic signal based on one or more characteristics of light sensed in the interior of the fluid delivery cannula when the fluid from the fluid source passes therethrough, and a controller including a processor and a non-transitory, processor readable storage medium, the non-transitory, processor readable storage medium comprising programming instructions stored thereon that, when executed, cause the processor to receive the electronic signal from the optical sensor, determine, based on the electronic signal, the one or more characteristics of the light, determine, based on the one or more characteristics of the light, a fluid infiltration event at the distal end of the fluid delivery cannula, and provide an alert indicating the fluid infiltration event.

In yet another embodiment, a method for detecting fluid infiltration includes causing fluid to move to a distal end of a fluid delivery cannula to a target site, sensing, via an optical sensor, light emitted from the target site and carried through at least a portion of the fluid delivery cannula, measuring one or more characteristics of the light, and determining a fluid infiltration event based on the one or more characteristics of the light.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an illustrative system for detecting fluid infiltration according to one or more embodiments shown and described herein;

FIG. 2 schematically depicts an illustrative section of a fluid delivery cannula, an illustrative optical sensor, and an illustrative controller of a system for detecting fluid infiltration according to one or more embodiments shown and described herein;

FIG. 3 schematically depicts an illustrative outer cover disposed relative to a fluid delivery of a system for detecting fluid infiltration according to one or more embodiments shown and described herein;

FIG. 4 schematically depicts an illustrative light emitting device of a system for detecting fluid infiltration according to one or more embodiments shown and described herein;

FIG. 5 schematically depicts a plurality of light emitting devices of a system for detecting fluid infiltration according to one or more embodiments shown and described herein;

FIG. 6 schematically depicts an illustrative beam stop of a system for detecting fluid infiltration according to one or more embodiments shown and described herein;

FIG. 7 schematically depicts an optical cover placed at the proximal end of the fluid delivery cannula according to one or more embodiments shown and described herein;

FIG. 8 schematically depicts an illustrative fluid source of a system for detecting fluid infiltration according to one or more embodiments shown and described herein;

FIG. 9 schematically depicts an illustrative valve and controller of a system for detecting fluid infiltration according to one or more embodiments shown and described herein;

FIG. 10 depicts a flowchart of an illustrative method for detecting fluid infiltration according to one or more embodiments shown and described herein;

FIG. 11 depicts a flowchart of another illustrative method for detecting fluid infiltration according to one or more embodiments shown and described herein; and

FIG. 12 depicts a flowchart of another illustrative method for detecting fluid infiltration according to one or more embodiments shown and described herein.

### DETAILED DESCRIPTION

Embodiments disclosed herein include devices, systems, and methods for detecting a fluid infiltration event. An infiltration event, as used herein, is the pooling of fluid in the soft tissue surrounding an intended target (e.g., a vein). Such pooling of fluid may be indicative of an improperly placed needle, leakage of bodily fluid, and/or one or more other undesirable circumstances for which remedial action may be necessary. When fluid is delivered to a subject, it is often desirable to quickly detect the infiltration event so that the remedial action can be quickly undertaken, such as, for example stopping the flow of fluid. In conventional systems, infiltration is generally detected by equipment located at the injection site (e.g., within the subject's tissue and/or disposed on the subject's tissue) and includes the use of single use components which must be disposed of as medical waste after one use. The location of such systems can also be problematic because such components are intrusive to a subject, it can be difficult to insert components in the tissue, and there exists a risk that certain components may inadvertently be pulled out of or otherwise become dislodged from the tissue, rendering the components inoperable for infiltration detection.

The devices, systems, and methods address the above issues by including components that are not intrusive because they are placed a distance away from the subject, but are particularly configured and arranged so as to be effective in detecting and/or mitigating infiltration events. As will be described in greater detail herein, the devices, systems, and methods include a fluid delivery cannula which can be shaped and sized to move fluid to a subject. The fluid delivery cannula is coupled into a subject at a target site (e.g., fluid delivery site, fluid injection site, etc.), such as a subject's vein. Any fluid which is present at the target site has inherent properties that cause light to be reflected back along the fluid delivery cannula. The properties of the reflected light can be used to determine that an infiltration event has occurred. Accordingly, the devices, systems, and methods described herein further include an optical sensor positioned such that it is optically coupled to the fluid delivery cannula for the purposes of detecting characteristics of the light within the fluid delivery cannula. The optical sensor generates an electronic signal corresponding to the light detected and transmits the electronic signal to a controller, which interprets the electronic signal to determine if a fluid infiltration event has occurred at the target site. If the controller detects a fluid infiltration event, the controller stops the flow of fluid along the fluid delivery cannula and/or transmits an alert indicating that the fluid infiltration event has occurred. In some embodiments, one or more light emitting devices are optically coupled to the fluid delivery cannula to emit light through the fluid delivery cannula to the distal end thereof, which is then reflected at the distal end back towards the optical sensor, thereby providing and consistent and controllable means of determining and detecting infiltration events.

Various components of the system, such as optical sensor, light emitting device, beam stop, and a controller, may be mounted on a remote mounting assembly spaced a distance from the subject (e.g., not attached to or inserted within tissue of the subject). The system may further utilize the cannula to provide fluids to a subject in addition to serving as light guides into and out of the target site, which may reduce the total number of components needed to detect fluid infiltration. It should be appreciated that, by mounting the various components a distance from the subject, such components can be assembled before use, whereas conventional systems can only be readied for use after affixing to a subject. Further, since various components described herein are placed a distance from the subject (e.g., not placed on the subject or within tissue of the subject), the components are reusable as opposed to conventional systems in which components must be disposed of as medical waste after use because the components are attached to a subject.

The terms “coupled,” “fixed,” “attached to,” and the like refer to both direct coupling, fixing, or attaching, as well as indirect coupling, fixing, or attaching through one or more intermediate components or features, unless otherwise specified herein.

The singular forms “a”, “an”, and “the” include plural references unless the context clearly dictates otherwise.

The term “at least one of” in the context of, e.g., “at least one of A, B, and C” refers only A, only B, only C, or any combination of A, B, and C.

As used in this application, stating that any part is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for case of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly that might, for example, otherwise share a same name.

Approximating language, as used herein throughout the specification and claims, is applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a ten percent margin.

As used herein, the term “adjacent” generally means two components being situated near, close to, or adjoining one another. That is, two components in direct contact with one another are considered to be adjacent in some embodiments. However, it should be appreciated that in other embodiments, components may be adjacent to one another without being in direct contact with one another.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

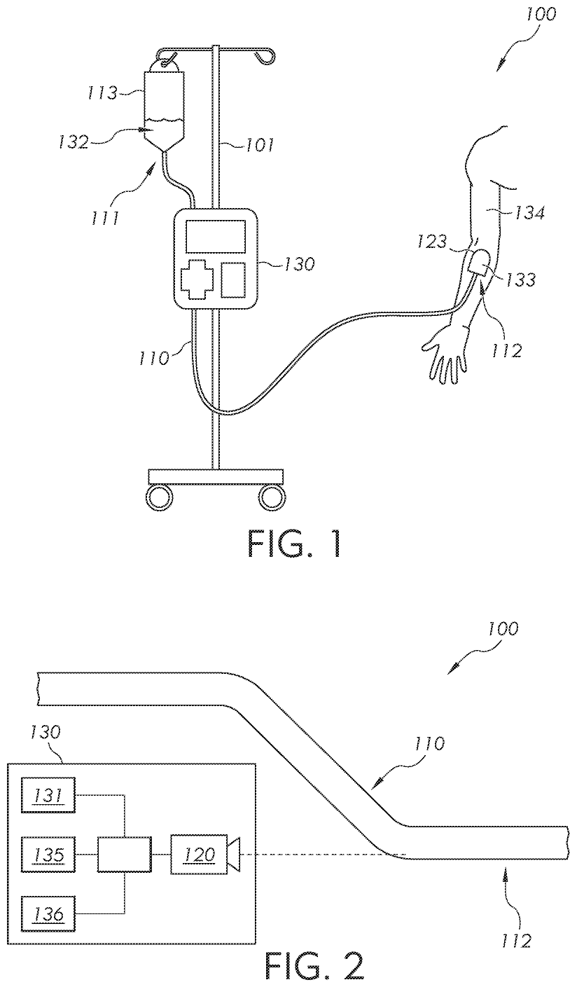

Referring now to FIG. 1, an embodiment of the system 100 is shown. The system 100 includes a fluid delivery cannula 110 including a proximal end 111 and a distal end 112 spaced a distance from the proximal end 111. The proximal end 111 of the fluid delivery cannula 110 is generally coupled to a fluid source, such as a fluid reservoir 113, as described in greater detail herein. The distal end 112 of the fluid delivery cannula 110 may be removably attached to a target site such as a target site 133 of a subject 134 (e.g., tissue of the subject 134 at a site of fluid injection, fluid delivery, or the like). Also depicted in FIG. 1 is a controller 130 that is coupled to the fluid delivery cannula 110 and configured to start fluid flow (e.g., cause the fluid 132 to flow from the fluid reservoir 113 to the target site 133), stop fluid flow (e.g., cause the fluid 132 to cease flowing from the fluid reservoir 113 to the target site 133), determine one or more characteristics of light within the fluid delivery cannula, provide alerts, and/or the like, as described in greater detail herein. Also depicted in FIG. 1 is an optical cover 123 disposed over the target site 133 to shield the target site 133 and the distal end 112 of the fluid delivery cannula 110 from ambient light, as described in greater detail herein.

The fluid delivery cannula 110 is generally shaped and sized to form a channel for passing the fluid 132 between the ends thereof (e.g., from the fluid reservoir 113 at the proximal end 111 to the target site 133 at the distal end 112). The fluid 132 may be a medication, a supplement, saline, or any other type of fluid that may be injected into a subject 134. The fluid delivery cannula 110 may be a flexible material such that it can be placed in any configuration between the proximal end 111 of the fluid delivery cannula 110 and the distal end 112 of the fluid delivery cannula 110. The fluid delivery cannula 110 may include any number of cross-sectional shapes, including but not limited to circular, rectangular, or any other shapes. The fluid delivery cannula 110 may include any number of cross-sectional dimensions, including, but not limited to, a diameter of about 0.1 millimeters, about 0.5 millimeters, about 2 millimeters, about 5 millimeters, about 10 millimeters, about 20 millimeters, or any range or value between any two of these values (including endpoints), or any other suitable size for fluid delivery to the subject 134.

In addition to carrying fluids, the fluid delivery cannula 110 may be formed of materials that allow the fluid delivery cannula to act as a light guide. That is, the fluid delivery cannula 110 may direct light proximally from the distal end 112 of the fluid delivery cannula 110 (e.g., from the target site 133). The materials used to form the fluid delivery cannula 110 may thus be any materials that allow for total internal reflection so as to propagate the light proximally from the distal end 112 of the fluid delivery cannula 110. Illustrative materials include, but are not limited to, polyvinyl chloride (PVC), silicone, glass, lucite, thermoplastic elastomer (TPE), acrylonitrile butadiene styrene (ABS), polyvinyl alcohol (PVA) and/or the like which may exhibit frequency doubling, raman scattering, and/or non-linear optical properties.

Still referring to FIG. 1, the system 100 may also include a support assembly 101 that supports various components of the system 100. For example, the support assembly 101 may support the fluid reservoir 113, the controller 130, and/or at least a portion of the fluid delivery cannula 110 thereon. That is, the fluid delivery cannula 110 may be routed such that a portion of the fluid delivery cannula 110 is coupled to the support assembly 101. In some embodiments, the support assembly 101 may be an IV pole. The support assembly 101 may include a number of holes, slots, brackets, hooks, or any other mounting feature so as to allow one or more components to be coupled to the support assembly 101. A variety of different types of components described in more detail below may be mounted to the support assembly 101. As depicted in FIG. 1, the support assembly 101 is generally located a distance from the target site 133. In some embodiments, the support assembly 101 may be configured so as to supply the fluid 132 from the fluid reservoir 113 via gravity feed and/or pump feed, as described in greater detail herein.

The fluid reservoir 113 is generally any device or component capable of containing a fluid to be delivered via the fluid delivery cannula 110 and is otherwise not limited by the present disclosure. For example, the fluid reservoir 113 may be an IV bag or the like that is fluidly coupled to the proximal end 111 of the fluid delivery cannula 110 such that fluid can flow from the fluid reservoir 113 through the fluid delivery cannula.

The distal end 112 of the fluid delivery cannula 110 is configured to be attached to a subject 134 at a target site 133 to allow fluid 132 to flow into the subject 134 (e.g., inserted within tissue of the subject 134). As such, the fluid delivery cannula 110 may include a piercing tip or the like on the distal end 112 thereof for piercing tissue of the subject 134 to insert at least a portion of the fluid delivery cannula 110 into the tissue of the subject 134. For example, a piercing tip of the fluid delivery cannula 110 may be inserted into circulatory tissue of the subject 134, such as a vein of the subject 134. In some instances, it may be desirable to ensure the distal end 112 of the fluid delivery cannula 110 is appropriately inserted in the tissue of the subject 134. For example, the fluid delivery cannula 110 may be appropriately inserted when the fluid delivery cannula 110 pierces vasculature of the subject 134 (e.g., a vein) at a single point. In another example, the fluid delivery cannula 110 may be inappropriately inserted when the fluid delivery cannula 110 pierces vasculature of the subject 134 (e.g., a vein) at a plurality of points or when the fluid delivery cannula 110 is inserted into tissue of subject 134 that is not vasculature (e.g., fails to pierce a vein). It should be appreciated that appropriate placement of the distal end 112 of the fluid delivery cannula 110 is such that blood flow within tissue of the subject 134 carries the fluid 132 from the fluid delivery cannula 110 away from the target site 133. In contrast, it should be appreciated that an inappropriate placement of the distal end 112 of the fluid delivery cannula 110 is such that fluid from the fluid delivery cannula 110 is not carried away from the target site 133, which can result in pooling of fluid at the target site 133 (e.g., an infiltration event). Pooling of fluid is detectable based on particular characteristics of light that is reflected from target site 133. That is, pooling of fluid at the target site 133 (e.g., an infiltration event) causes reflection of light that is sensed to have a relatively higher illuminance and/or intensity as compared to fluid that is not pooled at the target site 133.

The controller 130 generally includes various components for sensing the amount of light reflected from the target site 133 and determining whether an infiltration event has occurred. In addition, the controller 130 includes components for providing an alert regarding an infiltration event and/or controlling fluid flow. For example, referring now to FIG. 2, an embodiment of the system 100 is shown. More specifically, as shown in FIG. 2, the controller 130 includes an optical sensor 120, user interface hardware 131, a processor 135, and/or a non-transitory, processor readable storage medium 136. The controller 130 may also have other components not described herein.

The user interface hardware 131 is generally hardware utilized by the controller 130 to receive one or more inputs from a user and/or to provide one or more outputs to a user. For example, the user interface hardware 131 may include one or more input devices, such as, but not limited to, buttons, a touch screen, gesture capturing hardware, a microphone, a mouse, a keyboard, and/or the like. In another example, the user interface hardware 131 may include one or more output devices, such as, but not limited to, a display, a speaker, and/or the like. In some embodiments, the user interface hardware 131 may be a component that is used for both providing and receiving inputs, such as a touch screen display.

The optical sensor 120 is generally a device that is configured to sense one or more characteristics of light, particularly light reflected from the target site 133 (FIG. 1). Accordingly, the optical sensor 120 may be positioned such that the optical sensor 120 is optically coupled to the fluid delivery cannula 110 to detect light within the fluid delivery cannula 110, particularly reflected light from the target site 133 (FIG. 1). In order to sense the reflected light as it travels through the fluid delivery cannula, the optical sensor 120 may typically be physically coupled at any location between the proximal end 111 and the distal end 112 of the fluid delivery cannula 110. However, it should be understood that the optical sensor 120 may also be physically located at a distance apart from the fluid delivery cannula 110 but still optically coupled to the fluid delivery cannula 110 in other embodiments. In order for the optical sensor 120 to avoid contact with a subject (and thus avoid needing to be sanitized or disposed of between uses), the optical sensor 120 may be physically located a distance from the subject 134 (FIG. 1). For example, as depicted in FIG. 1, the optical sensor 120, as part of the controller 130, is coupled to the support assembly 101. While FIG. 2 depicts the optical sensor 120 as being housed within the controller 130, it should be appreciated that this is merely illustrative. That is, in other embodiments, the optical sensor 120 may be housed outside of the controller 130 and communicatively coupled to the controller 130 such that signals may be communicated between the controller 130 and the optical sensor 120, as described in greater detail herein.

The optical sensor 120 may be any number of different types of sensors that are capable of detecting various wavelengths of light, particularly sensors that may be adapted for low-light level photodetection. In one embodiment, the optical sensor 120 includes a silicon photomultiplier avalanche diode (SPAD). The optical sensor 120 may also include any other number of types of optical sensors, camera, and/or the like. The optical sensor 120 may also be adapted to detect light from any wavelength, including infrared light, visible light, or any other wavelength of light.

As discussed herein, the optical sensor 120 is communicatively coupled to the controller 130. That is, the optical sensor 120 is coupled to transmit one or more electronic signals to the controller 130, the one or more electronic signals being generated by the optical sensor 120 to correspond to one or more characteristics of the sensed light that is reflected from the target site 133 (FIG. 2). The one or more electronic signals are received by the controller 130 and used to determine whether an infiltration event has occurred. The controller 130 may utilize the processor 135 and/or the processor readable storage medium 136 for the purposes of determining whether an infiltration event has occurred. For example, the processor readable storage medium 136 may store programming instructions thereon that, when executed by the processor 135, cause the processor to receive the electronic signals, interpret the electronic signals, and make a determination based on the interpreted electronic signals.

For example, the optical sensor 120 may detect first characteristics of light when the fluid delivery cannula 110 is placed into tissue and prior to fluid movement through the fluid delivery cannula 110 so as to establish a baseline reading. The baseline reading may be automatically triggered (e.g., by sensing that the fluid delivery cannula 110 has been inserted) or manually triggered (e.g., by receiving an input from a user via the user interface hardware 131 indicating that the fluid delivery cannula 110 has been inserted). The detected first characteristics of the light may be encoded and transmitted to the controller 130 as an electronic signal. The optical sensor 120 may then detect second characteristics of light when fluid is directed from the proximal end 111 of the fluid delivery cannula 110 to the distal end 112 of the fluid delivery cannula 110 (e.g., when the fluid is directed to the target site 133). The second reading may be automatically triggered (e.g., triggered when fluid is allowed to flow through the fluid delivery cannula 110) or manually triggered (e.g., by receiving a second input from a user via the user interface hardware 131 indicating that fluid flow to the target site 133 is occurring). The detected second characteristics of the light may be encoded and transmitted to the controller 130 as another electronic signal. The controller 130, utilizing the processor readable storage medium 136 and the processor 135, compares the second characteristics to the first characteristics. Alternatively, the controller 130 may utilize a database (not shown) including a look up table or the like that contains electronic signal references that correspond to various light characteristics and are usable to correlate the signal with the detected light characteristics.

When the fluid 132 is delivered to the subject 134, the fluid 132 is typically carried away from the target site 133 by the blood flow of the subject 134 when the cannula is appropriately placed. This leaves relatively little to no fluid 132 at the target site 133 as the fluid 132 is being constantly moved away from the target site 133. Since there is little to no fluid 132 at the target site 133 when the fluid 132 is delivered to a subject 134 under appropriate placement, there is relatively less light reflected from the target site 133 along the fluid delivery cannula 110. In contrast, when the fluid 132 is delivered to the subject 134 under inappropriate placement, the fluid 132 may infiltrate the tissue of the subject 134 surrounding the target site 133, and/or the fluid 132 may remain at or near the target site 133. This results in a greater amount of fluid 132 at or near the target site 133 compared to when the fluid 132 is delivered under appropriate placement. Since more fluid 132 is located at the target site 133, the brightness and/or intensity of light reflected from the target site 133 by the fluid 132 and guided along the fluid delivery cannula 110 may be greater than when no fluid infiltration occurs.

If the second characteristics of the sensed light indicate brighter and/or more intense light than the first characteristics of the sensed light, the controller 130, based on the corresponding electronic signals received from the optical sensor 120 detecting the light, can determine that infiltration has occurred. In other embodiments, if the second characteristics of the sensed light indicate substantially equal to or less brightness and/or intensity of the light, the controller 130, based on the corresponding electronic signals received from the optical sensor 120, can determine that infiltration has not occurred. For example, if the second characteristics indicate light being about one-percent brighter and/or more intense, about five-percent brighter and/or more intense, about ten-percent brighter, or greater than the first characteristics of light, the controller 130 determines that infiltration event has occurred.

As described in greater detail herein, if infiltration is detected, the controller may cause the user interface hardware 131 to provide an output. For example, the user interface hardware 131 may be instructed to emit a light, emit an audible sound, emit a vibration, and/or the like.

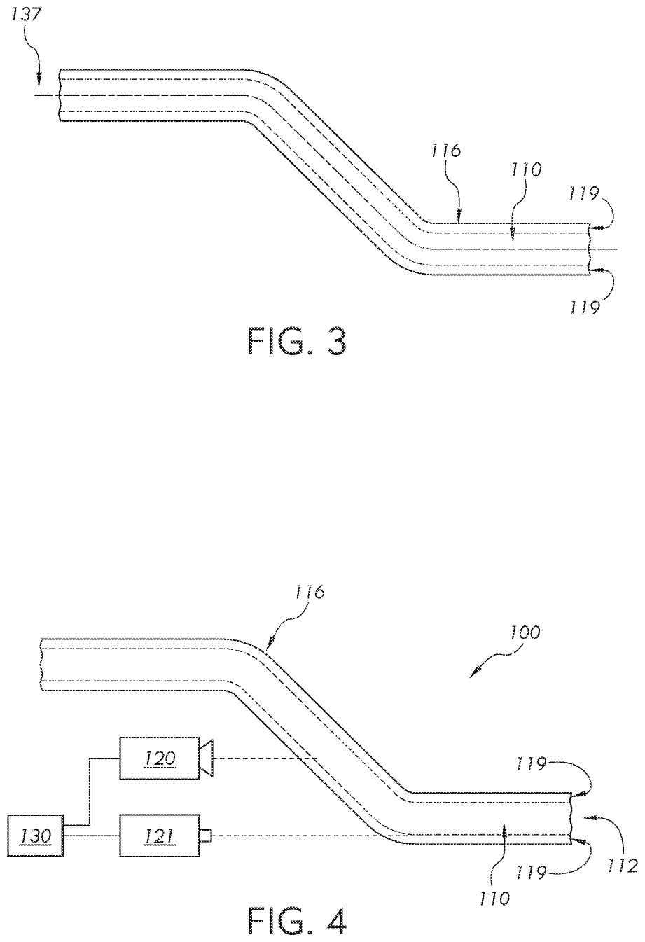

In some embodiments, the system 100 may include additional components to facilitate light propagation in order to ensure an appropriate measurement of the light is obtained by the optical sensor 120 for the purposes of determining whether an infiltration event has occurred. Referring now to FIG. 3, an outer cover 116 may be disposed around the fluid delivery cannula 110 such that an enclosure 119 is formed between the fluid delivery cannula 110 and the outer cover 116 in some embodiments. The outer cover 116 generally includes a reflective interior surface (e.g., a reflective surface that faces the fluid delivery cannula 110) to further ensure total internal reflection of light through the fluid delivery cannula 110. It should be appreciated that the characteristics and positioning of the outer cover 116 around the fluid delivery cannula 110 allows for additional control over the light that is reflected and propagated. In some embodiments, the outer cover 116 may completely cover the fluid delivery cannula 110. In other embodiments, the outer cover 116 may cover only a portion of the fluid delivery cannula 110 (e.g., the outer cover may extend over the fluid delivery cannula 110 between the distal end 112 thereof and the location of the optical sensor (FIG. 2). Still referring to FIG. 3, the outer cover 116 may include cross sectional shape, including, but not limited to circular or rectangular. The outer cover 116 may have the same cross sectional shape as the fluid delivery cannula 110, or the outer cover 116 may have a different cross sectional shape as the fluid delivery cannula 110. The outer cover 116 may be constructed of polyvinyl chloride (PVC), silicone, glass, lucite, thermoplastic elastomer (TPE), acrylonitrile butadiene styrene (ABS), polyvinyl alcohol (PVA) and/or the like which may exhibit frequency doubling, raman scattering, and/or non-linear optical properties. In some embodiments, the outer cover 116 may be deposited on the fluid delivery cannula 110 (e.g., painted, sputtered on, or the like). In other embodiments, the outer cover 116 may be wrapped around the fluid delivery cannula 110. The outer cover 116 may be permanently fixed to the fluid delivery cannula 110 or may be removably attached to the fluid delivery cannula 110.

Another illustrative component for facilitating light propagation is depicted in FIG. 4. More specifically, the system 100 further includes a light emitting device 121 that is optically coupled to the fluid delivery cannula 110 such that light emitted from the light emitting device 121 is propagated toward the distal end 112 of the fluid delivery cannula 110 onto the target site 133. That is, the light emitting device 121 generally provides additional light that is used for the purposes of determining characteristics of reflected light as described herein. As depicted in FIG. 4, in some embodiments, the fluid delivery cannula 110 may further include the outer cover 116 in addition to the light emitting device 121 such that light from the light emitting device and/or reflected light are propagated by the enclosure 119 between the outer cover 116 and the fluid delivery cannula 110. The light emitting device 121 may emit light along the enclosure 119 towards the distal end 112 of the fluid delivery cannula 110, such that the enclosure 119 operates as a light guide to propagate the light from the light emitting device 121 towards the distal end 112 of the fluid delivery cannula 110. The light emitting device 121 may be optically coupled to the fluid delivery cannula 110 at any location between the distal end 112 and the proximal end 111 (FIG. 1) of the fluid delivery cannula 110. The light emitting device 121 may be any type of light emitting device, including but not limited to a laser diode, a visible light emitting device, an infrared light emitting device, or any other type of light emitting device.

In some embodiments, the light emitting device 121 may be communicatively coupled to the controller 130. The controller 130 may automatically activate the light emitting device 121 when a measurement of light is to occur. The controller 130 further may activate and deactivate the light emitting device 121 at regularly timed intervals, such as every second, every five seconds, or any other interval. The controller 130 further may activate and deactivate the light emitting device 121 at irregular intervals. The controller 130 may further control various characteristics of the light emitted by the light emitting device 121, such as brightness, intensity, wavelength, or the like, so as to ensure particular characteristics of reflected light for the purposes of measuring the reflected light as described herein.

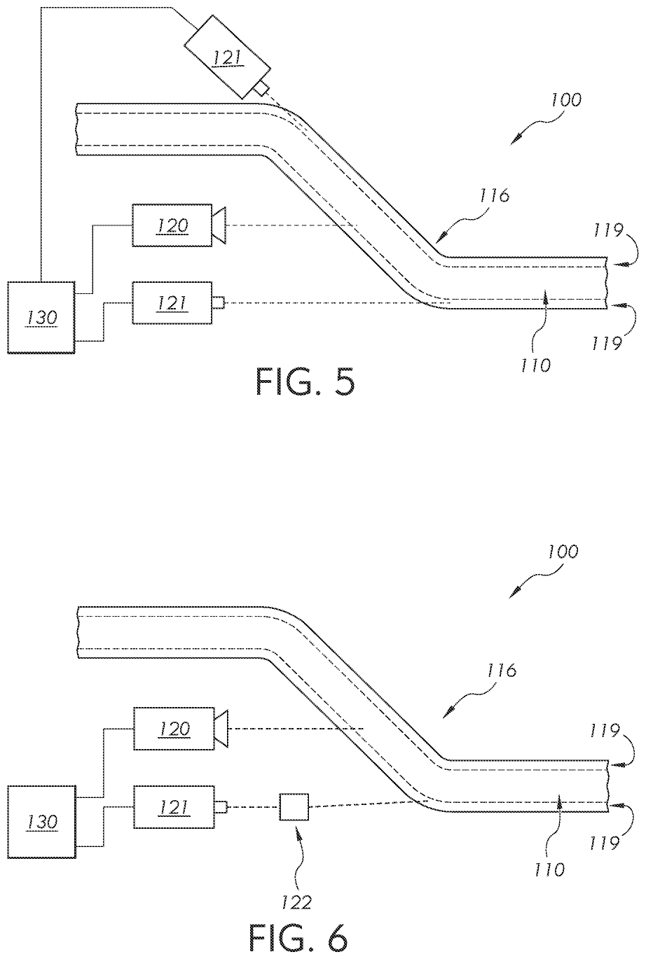

In some embodiments, a plurality of light emitting devices 121 may be utilized, as depicted in FIG. 5. The plurality of light emitting devices 121 may be optically coupled to the fluid delivery cannula 110 such that light emitted from the light emitting devices 121 is propagated toward the distal end 112 of the fluid delivery cannula 110 onto the target site 133. The plurality of light emitting devices 121 may emit light along the enclosure 119 towards the distal end 112 of the fluid delivery cannula 110, such that the enclosure 119 operates as a light guide to propagate the light from the plurality of light emitting devices 121 towards the distal end 112 of the fluid delivery cannula 110. The plurality of light emitting devices 121 may be optically coupled to the fluid delivery cannula 110 at any location between the distal end 112 and the proximal end 111 (FIG. 1) of the fluid delivery cannula 110. The plurality of light emitting devices 121 may all be activated to propagate light along the fluid delivery cannula 110, or only a portion of the plurality of light emitting devices 121 may be activated to propagate light along the fluid delivery cannula 110. An operator may select how many of the plurality of light emitting devices 121 to be activated in order to more precisely control the amount of light propagated along the fluid delivery cannula 110. The plurality of light emitting devices 121 may be any type of light emitting device, including but not limited to a laser diode, a visible light emitting device, an infrared light emitting device, or any other type of light emitting device. In some embodiments, the plurality of light emitting devices 121 may be all of the same type of light emitting devices, or may be a mixed type of light emitting devices. An operator may select which type of light emitting device is activated so the preferred type of light in any given situation may be propagated along the fluid delivery cannula 110.

In some embodiments, the plurality of light emitting devices 121 may be communicatively coupled to the controller 130. The controller 130 may automatically activate the plurality of light emitting devices 121 when a measurement of light is to occur. The controller 130 may activate or deactivate all or a portion of the plurality of light emitting devices 121. The controller 130 may activate or deactivate a specific type of light emitting device from among the plurality of light emitting devices The controller 130 further may activate and deactivate the plurality of light emitting devices 121 at regularly timed intervals, such as every second, every five seconds, or any other interval. The controller 130 further may activate and deactivate the light emitting device 121 at irregular intervals. The controller 130 may further control various characteristics of the light emitted by the light emitting device 121, such as brightness, intensity, wavelength, or the like, so as to ensure particular characteristics of reflected light for the purposes of measuring the reflected light as described herein.

In some embodiments, the light to be reflected at the target site 133 may have particular characteristics in order to obtain a sufficient reading at the optical sensor 120. As such, one or more optical elements may be used to particularly tune the light emitted from the light emitting devices 121 accordingly. For example, referring now to FIG. 6, the system 100 may include a beam stop 122 or the like that is optically positioned along a path between the light emitting device 121 and the fluid delivery cannula 110. While FIG. 6 depicts a single beam stop 122 and a single light emitting device 121, any number of beam stops 122 and/or light emitting devices 121 may be used, including a larger number of beam stops 122 than light emitting devices 121 such that a plurality of beam stops 122 are used with a single light emitting device 121.

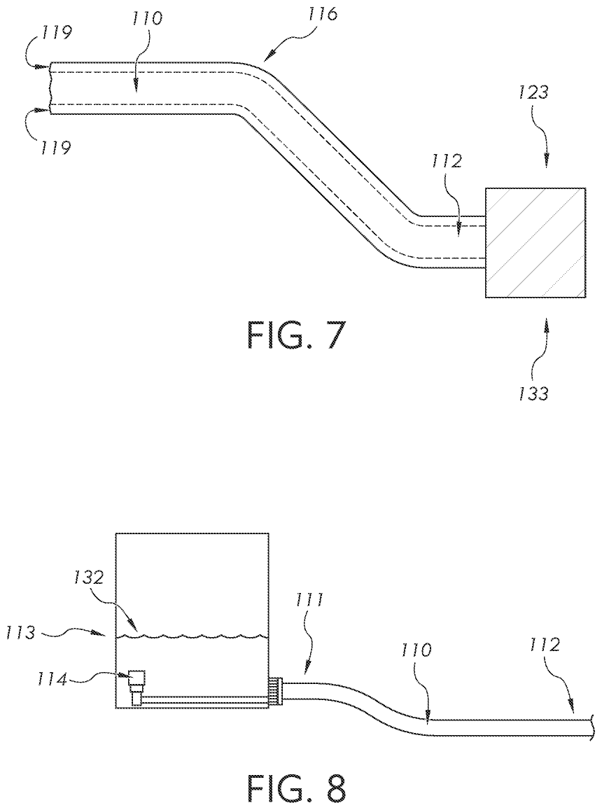

In some embodiments, additional components may be used to ensure appropriate reflectance of light propagated through the fluid delivery cannula 110 and/or to ensure that light is appropriately reflected by fluid when present. For example, referring now to FIG. 7, an optical cover 123 is depicted at the distal end 112 of the fluid delivery cannula 110 and/or located partially over the target site 133. The optical cover 123 includes a material that does not allow for the passage of light therethrough, such as plastic or metal, or any other appropriate material. The optical cover 123 may be any suitable shape, including but not limited to a square, a circle, a rectangle, or an irregular shape. The optical cover 123 may be appropriately sized so as to cover the distal end 112 of the fluid delivery cannula 110, a distal end of the outer cover 116, and/or the target site 133.

Still referring to FIG. 7, the optical cover 123 may shield the distal end 112 of the fluid delivery cannula 110 and/or the target site 133 from external light (e.g., light that is not propagated through the fluid delivery cannula for the purposes of reflecting on any fluid present at the target site 133 as described herein). In some embodiments, the optical cover 123 may be bonded to the subject 134 (FIG. 1) with adhesive or any other suitable bonding agent to seal the optical cover 123 to the subject 134 and further block external light sources. In other embodiments, the optical cover 123 may be deformable such that the optical cover can be adjusted to the contours of the subject 134 (FIG. 1) to further seal the optical cover 123 to the subject 134 and block external light sources.

By shielding the distal end 112 of the fluid delivery cannula 110 and/or the target site 133 from external light sources, light reflected at the target site 133 may be primarily from the light emitting device 121 (FIG. 6). This may allow for increased control of the characteristics of the light, which may enhance the efficiency and accuracy of the ability of the system 100 to detect a fluid infiltration event compared to an embodiment of the system without an optical cover 123.

FIG. 8 depicts the fluid reservoir 113 fluidly coupled to the proximal end 111 of the fluid delivery cannula 110. As previously described herein, the fluid reservoir 113 houses the fluid 132 to be delivered to the target site 133 (FIG. 1). Referring also to FIG. 1, in some embodiments, the proximal end 111 of the fluid delivery cannula 110 is placed at a higher elevation than the distal end 112, such that the fluid 132 flows under force of gravity from the proximal end 111 of the fluid delivery cannula 110 towards the distal end 112.

However, in some embodiments, a pump 114 may be utilize to push the fluid 132 to flow from the proximal end 111 of the fluid delivery cannula 110 towards the distal end 112. While FIG. 8 depicts the pump 114 as being disposed within the fluid reservoir 113, the present disclosure is not limited to such a location. That is, the pump 114 may be disposed at any location between the proximal end 111 and the distal end 112 of the fluid delivery cannula 110. For example, the pump 114 may be located within the controller 130 in some embodiments. The pump 114 may be communicatively coupled to the controller 130 such that activation and deactivation of the pump 114 is directed by the controller 130. That is, the controller 130 can transmit an activation signal to the pump 114 to cause the pump 114 to pump fluid and can also transmit a deactivation signal to the pump 114 to cause the pump 114 to cease pumping fluid. The pump 114 is otherwise not limited by the present disclosure and can be any type of fluid pump, including, but not limited to, dynamic pumps such as centrifugal pumps and vertical turbine pumps, and/or positive displacement pumps such as reciprocating pumps and rotary pumps.

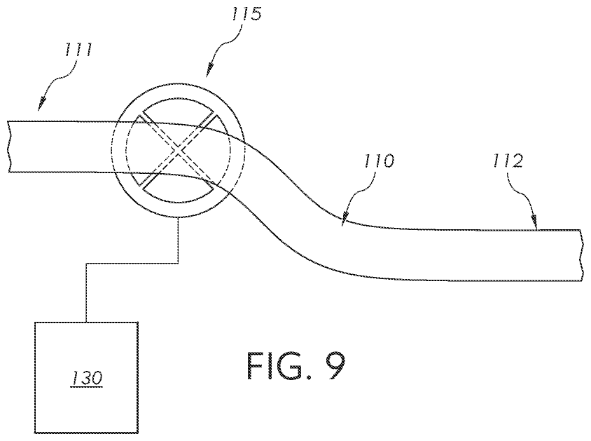

In addition to the pump 114 or in lieu of the pump 114, other components may be used to control fluid flow. For example, as depicted in FIG. 9, the system 100 may further include a valve 115. The valve 115 is generally disposed along the fluid delivery cannula 110 at a location between the proximal end 111 and the distal end 112 of the fluid delivery cannula 110. The valve 115 may be placed at any location between the proximal end 111 and the distal end 112. The valve 115 may be communicatively coupled to the controller 130 such that activation and deactivation of the valve 115 is directed by the controller 130. That is, the controller 130 can transmit an open signal to the valve 115 to cause the valve 115 to open and allow fluid to pass therethrough and can also transmit a close signal to the valve 115 to cause the valve 115 to close, thereby blocking fluid from passing therethrough. The valve 115 is otherwise not limited by the present disclosure and can be any type of valve, including, but not limited to, isolation valves, regulation valves, relief valves, non-return valves, and/or the like.

While FIGS. 8 and 9 respectively depict a single pump and a single valve, it should be appreciated that the present disclosure is not limited to such embodiments. That is, any number of pumps and/or valves may be incorporated without departing from the scope of the present disclosure.

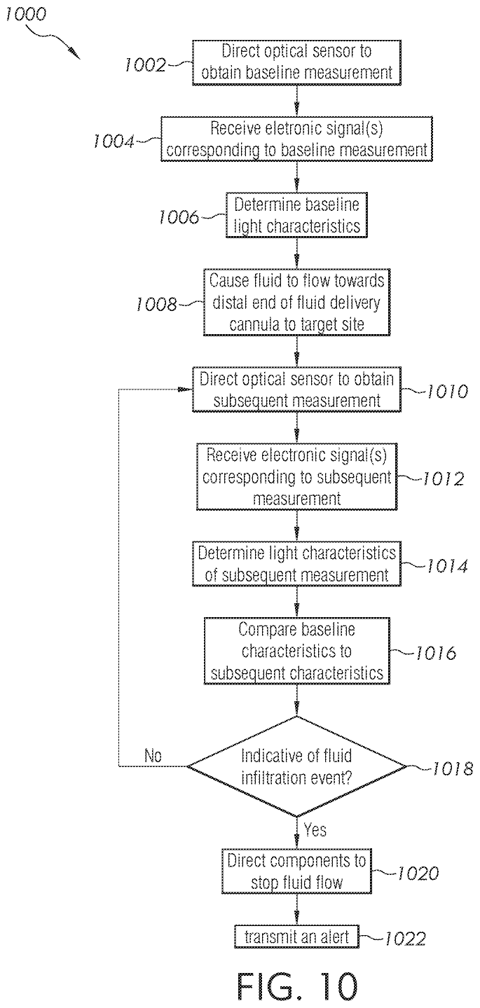

Referring now to FIG. 10, the steps of a method for detecting fluid infiltration is described. With reference also to FIGS. 1-4 and FIGS. 8-9, at block 1002, the method 1000 includes directing the optical sensor 120 to obtain a baseline measurement of the brightness and/or intensity of light within the fluid delivery cannula 110. At block 1004, the method 1000 includes receiving, from the optical sensor 120, an electronic signal of the baseline measurement of light characteristics (e.g., brightness and/or intensity of light) within the fluid delivery cannula 110. That is, the method 1000 includes sensing, via the optical sensor, light emitted from the target site and carried through at least a portion of the fluid delivery cannula. At block 1006, the method 1000 includes determining a baseline light characteristics (e.g., light brightness and/or intensity of the light) detected by the optical sensor 120 from within the fluid delivery cannula 110. At block 1008, the method 1000 includes causing fluid to flow along the fluid delivery cannula 110 towards the distal end 112 of the fluid delivery cannula and towards the target site 133. At block 1010, the method 1000 includes directing the optical sensor 120 to obtain a subsequent measurement of light characteristics (e.g., brightness and/or intensity of the light) along the fluid delivery cannula 110. At block 1012, the method 1000 includes receiving from the optical sensor 120 an electronic signal of the subsequent measurement of the characteristics of the light (e.g., brightness and/or intensity of light) within the fluid delivery cannula 110. At block 1014, the method 1000 includes determining a subsequent light brightness and/or intensity of the light detected by the optical sensor 120 from within the fluid delivery cannula 110. At block 1016, the method 1000 includes comparing the baseline brightness and/or intensity of light within the fluid delivery cannula 110 to the subsequent brightness and/or intensity of light within the fluid delivery cannula. At block 1018, the method 1000 includes a step of determining if a fluid infiltration event has occurred based on the outcome of comparing the baseline and subsequent light characteristics (e.g., brightness and/or intensity of light) within the fluid delivery cannula 110. For example, a determination that a fluid infiltration event has occurred may be if the characteristics of the subsequent light measurement differ from the baseline characteristics by a certain percentage (e.g., at least a 5% difference). In another example, a determination that a fluid infiltration event has occurred may be if the characteristics of the subsequent light measurement is in any way different (e.g., brighter, more intense, and/or the like). If it is determined that a fluid infiltration has occurred (block 1018=“YES”), the method proceeds to the step described at block 1020. If it is determined that a fluid infiltration has not occurred (block 1018=“NO”), the method proceeds to the step described at block 1010. At block 1020, the method 1000 includes directing components to stop the fluid flow along the fluid delivery cannula 110. The components used to stop the fluid flow may be the valve 115 placed along the fluid delivery cannula 110 and/or the pump 114 used to flow fluid along the fluid delivery cannula 110. Such components may be directed by transmitting a signal to the components, as described herein. At block 1022, the method 1000 includes transmitting an alert with the user interface hardware 131.

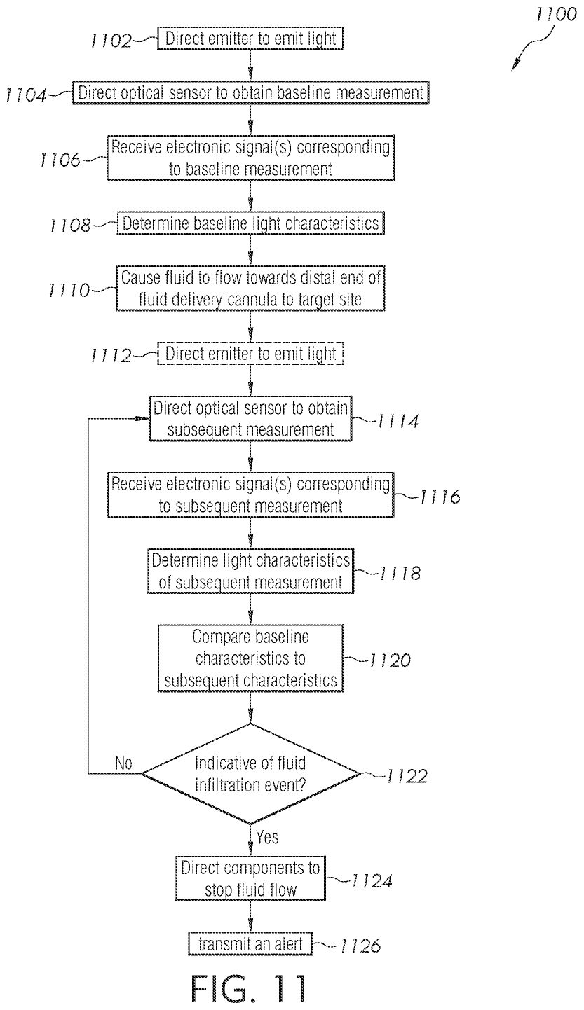

Referring now to FIG. 11, the steps of a method for detecting fluid infiltration is described. With reference also to FIGS. 1-4 and FIGS. 8-9, the method 1100 at block 1102 includes directing the light emitting device 121 to emit light along the fluid delivery cannula 110. At block 1104, the method 1100 includes directing the optical sensor 120 to obtain a baseline measurement of light characteristics (e.g., brightness and/or intensity of light) within the fluid delivery cannula 110. At block 1106, the method 1100 includes receiving, from the optical sensor 120, an electronic signal of the baseline measurement of light characteristics (e.g., brightness and/or intensity of light) within the fluid delivery cannula 110. That is, the method 1100 includes sensing, via the optical sensor, light emitted from the target site and carried through at least a portion of the fluid delivery cannula. At block 1108, the method 1100 includes determining a baseline of light characteristics (e.g., light brightness and/or intensity of the light) detected by the optical sensor 120 from within the fluid delivery cannula 110. At block 1110, the method 1100 includes causing fluid to flow along the fluid delivery cannula 110 towards the distal end 112 of the fluid delivery cannula and towards the target site 133. At block 1112, the method 1100 optionally includes directing the light emitting device 121 to emit light along the fluid delivery cannula 110. At block 1114, the method 1100 includes directing the optical sensor 120 to obtain a subsequent measurement of light characteristics (e.g., brightness and/or intensity of the light) along the fluid delivery cannula 110. At block 1116, the method 1100 includes receiving from the optical sensor 120 an electronic signal of the subsequent measurement of light characteristics (e.g., brightness and/or intensity of light) within the fluid delivery cannula 110. At block 1118, the method 1100 includes determining a subsequent light characteristics (e.g., brightness and/or intensity of the light) detected by the optical sensor 120 from within the fluid delivery cannula 110. At block 1120, the method 1100 includes comparing the baseline light characteristics (e.g., brightness and/or intensity of light) within the fluid delivery cannula 110 to the subsequent light characteristics (e.g., brightness and/or intensity of light) within the fluid delivery cannula. At block 1122, the method 1100 includes a step of determining if a fluid infiltration event has occurred based on the outcome of comparing the baseline and subsequent light characteristics (e.g., brightness and/or intensity of light) within the fluid delivery cannula 110. For example, a determination that a fluid infiltration event has occurred may be if the characteristics of the subsequent light measurement differ from the baseline characteristics by a certain percentage (e.g., at least a 5% difference). In another example, a determination that a fluid infiltration event has occurred may be if the characteristics of the subsequent light measurement is in any way different (e.g., brighter, more intense, and/or the like). If it is determined that a fluid infiltration has occurred (block 1122=“YES”), the method proceeds to the step described at block 1124. If it is determined that a fluid infiltration has not occurred (block 1122=“NO”), the method proceeds to the step described at block 1114. At block 1124, the method 1100 includes directing components to stop the fluid flow along the fluid delivery cannula 110. The components used to stop the fluid flow may be the valve 115 placed along the fluid delivery cannula 110 and/or the pump 114 used to flow fluid along the fluid delivery cannula 110. Such components may be directed by transmitting a signal to the components, as described herein. At block 1126, the method 1100 includes transmitting an alert with the user interface hardware 131.

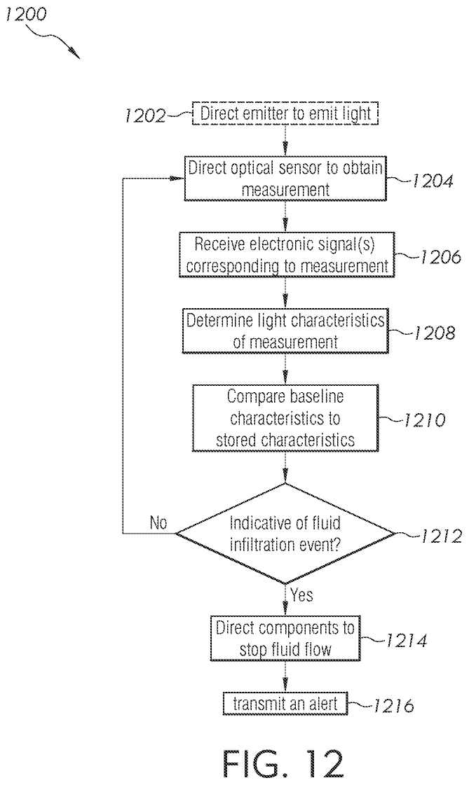

Referring now to FIG. 12 the steps of a method for detecting fluid infiltration is described. With reference also to FIGS. 1-4 and FIGS. 8-9, the method 1200 at block 1202 includes optionally directing the light emitting device 121 to emit light along the fluid delivery cannula 110. At block 1204, the method 1200 includes directing the optical sensor 120 to obtain a measurement of light characteristics (e.g., brightness and/or intensity of light) within the fluid delivery cannula 110. At block 1206, the method 1200 includes receiving, from the optical sensor 120, an electronic signal of the measurement of the brightness and/or intensity of light within the fluid delivery cannula 110. At block 1208, the method 1200 includes determining light characteristics (e.g., brightness and/or intensity of the light) detected by the optical sensor 120 from within the fluid delivery cannula 110. At block 1210, the method 1200 includes comparing the measured light characteristics (e.g., brightness and/or intensity of light) within the fluid delivery cannula 110 to previously stored light characteristics (e.g., brightness and/or intensity of light) within the fluid delivery cannula. At block 1212, the method 1200 includes determining if a fluid infiltration event has occurred based on the outcome of comparing the measured and previously stored light characteristics (e.g., brightness and/or intensity of light) within the fluid delivery cannula 110. If it is determined that a fluid infiltration has occurred (block 1212=“YES”), the method proceeds to the step described at block 1214. If it is determined that a fluid infiltration has not occurred (block 1212=“NO”), the method proceeds to the step described at block 1204. At block 1214, the method 1200 directing components to stop the fluid flow along the fluid delivery cannula 110. The components used to stop the fluid flow may be the valve 115 placed along the fluid delivery cannula 110 and/or the pump 114 used to flow fluid along the fluid delivery cannula 110. Such components may be directed by transmitting a signal to the components, as described herein. At block 1216, the method 1200 includes transmitting an alert with the user interface hardware 131.

Further aspects of the present disclosure are provided by the subject matter of the following clauses:

A system for detecting fluid infiltration, the system comprising: a fluid delivery cannula comprising a distal end and a proximal end, a fluid source fluidly coupled to the proximal end of the fluid delivery cannula, an optical sensor optically coupled to an interior of the fluid delivery cannula, wherein the optical sensor is disposed between the distal end and the proximal end of the fluid delivery cannula and configured to generate an electronic signal based on one or more characteristics of light sensed in the interior of the fluid delivery cannula when fluid from the fluid source is passed therethrough, and a controller configured to receive the electronic signal from the optical sensor and determine a fluid infiltration event based on the electronic signal.

The system for detecting fluid infiltration of any of the previous clauses, the system comprising: an outer cover at least partially surrounding the fluid delivery cannula, the outer cover comprising an interior reflective surface to internally reflect the light in the interior of the fluid delivery cannula.

The system for detecting fluid infiltration of any of the previous clauses, further comprising: a light emitting device optically coupled to the interior of the fluid delivery cannula and disposed between the distal end and the proximal end of the fluid delivery cannula, the light emitting device emitting light that is carried through the interior of the fluid delivery cannula.

The system for detecting fluid infiltration of any of the previous clauses, further comprising: the light emitting device is a laser diode.

The system for detecting fluid infiltration of any of the previous clauses, comprising: the light emitting device is a plurality of light emitting devices.

The system for detecting fluid infiltration of any of the previous clauses, comprising: a beam stop disposed between the light emitting device and the interior of the fluid delivery cannula, the beam stop configured to modify the light emitted from the light emitting device before the light is directed to the interior of the fluid delivery cannula.

The system for detecting fluid infiltration of any of the previous clauses, wherein the optical sensor is a silicon photomultiplier avalanche diode (SPAD).

The system for detecting fluid infiltration of any of the previous clauses, comprising: a pump fluidly coupled between the distal end of the fluid delivery cannula and the fluid source for pumping fluid from the fluid source to the proximal end of the fluid delivery cannula.

The system for detecting fluid infiltration of any of the previous clauses, wherein the fluid delivery cannula is a gravity fed fluid delivery cannula.

The system for detecting fluid infiltration of any of the previous clauses, comprising: a valve fluidly coupled to the fluid delivery cannula, the valve configured to control fluid movement into the fluid delivery cannula or through at least a portion of the fluid delivery cannula.

The system for detecting fluid infiltration of any of the previous clauses, wherein the valve is communicatively coupled to the controller such that the controller provides a signal to the valve to cause the valve to open or close.

The system for detecting fluid infiltration of any of the previous clauses, comprising: a user interface communicatively coupled to the controller.

The system for detecting fluid infiltration of any of the previous clauses, wherein the user interface is configured to provide an alert to a user when the fluid infiltration event is determined by the controller.

A fluid infiltration detection device, comprising: an optical sensor optically coupled to a fluid delivery cannula delivering fluid from a fluid source therethrough and positioned at a location remote from a distal end of the fluid delivery cannula, the optical sensor configured to generate an electronic signal based on one or more characteristics of light sensed in the interior of the fluid delivery cannula when the fluid from the fluid source passes therethrough, and a controller comprising a processor and a non-transitory, processor-readable storage medium, the non-transitory, processor-readable storage medium comprising programming instructions stored thereon that, when executed, cause the processor to: receive the electronic signal from the optical sensor, determine, based on the electronic signal, the one or more characteristics of the light, determine, based on the one or more characteristics of the light, a fluid infiltration event at the distal end of the fluid delivery cannula, and provide an alert indicating the fluid infiltration event.

The fluid infiltration detection device of any of the previous clauses, comprising: a light emitting device that emits light that is carried through an interior of the fluid delivery cannula, reflected at a target site adjacent to the distal end of the fluid delivery cannula, and sensed by the optical sensor.

The fluid infiltration detection device of any of the previous clauses, wherein the programming instructions that cause the processor to determine the fluid infiltration event further cause the processor to measure a brightness and/or intensity of the light at the distal end of the fluid delivery cannula.

A method for detecting fluid infiltration, the method comprising: causing fluid to move to a distal end of a fluid delivery cannula to a target site, sensing, via an optical sensor, light emitted from the target site and carried through at least a portion of the fluid delivery cannula, measuring one or more characteristics of the light, and determining a fluid infiltration event based on the one or more characteristics of the light.

The method of the previous clauses further comprising: measuring one or more baseline characteristics of the light prior to causing the fluid to move, wherein measuring the one or more characteristics of the light comprises measuring the one or more characteristics after causing the fluid to move.

The method of any of the previous clauses, further comprising: comparing the one or more baseline characteristics to the one or more characteristics, wherein determining the fluid infiltration event is based on the comparing.

The method of any of the previous clauses, further comprising: emitting light towards the distal end of the fluid delivery cannula such that the light reflects off a target site, wherein measuring the one or more characteristics of the light comprises measuring one or more characteristics of reflected light.

The method of any of the previous clauses, wherein causing the fluid to move comprises moving the fluid via a pump.

The method of any of the previous clauses, further comprising: instructing the pump to cease pumping the fluid upon a determination of the fluid infiltration event.

The method of any of the previous clauses, further comprising: instructing a valve fluidly coupled to the fluid delivery cannula to close upon a determination of the fluid infiltration event.

The method of any of the previous clauses, further comprising: transmitting a signal indicative of the fluid infiltration event.

What is claimed is:

1. A system for detecting fluid infiltration, the system comprising:
a fluid delivery cannula comprising a distal end and a proximal end;
a fluid source fluidly coupled to the proximal end of the fluid delivery cannula;
an optical sensor optically coupled to an interior of the fluid delivery cannula, wherein the optical sensor is disposed between the distal end and the proximal end of the fluid delivery cannula and configured to generate an electronic signal based on one or more characteristics of light sensed in the interior of the fluid delivery cannula when fluid from the fluid source is passed therethrough; and
a controller configured to receive the electronic signal from the optical sensor and determine a fluid infiltration event based on the electronic signal.

2. The system of claim 1, further comprising:
an outer cover at least partially surrounding the fluid delivery cannula, the outer cover comprising an interior reflective surface to internally reflect the light in the interior of the fluid delivery cannula.

3. The system of claim 1, further comprising:
a light emitting device optically coupled to the interior of the fluid delivery cannula and disposed between the distal end and the proximal end of the fluid delivery cannula, the light emitting device emitting light that is carried through the interior of the fluid delivery cannula.

4. The system of claim 3, wherein the light emitting device is a laser diode.

5. The system of claim 3, wherein the light emitting device is a plurality of light emitting devices.

6. The system of claim 3, further comprising:
a beam stop disposed between the light emitting device and the interior of the fluid delivery cannula, the beam stop configured to modify the light emitted from the light emitting device before the light is directed to the interior of the fluid delivery cannula.

7. The system of claim 1, wherein the optical sensor is a silicon photomultiplier avalanche diode (SPAD).

8. The system of claim 1, further comprising:
an optical cover disposed at the proximal end of the fluid delivery cannula.

9. The system of claim 1, further comprising:
a pump fluidly coupled between the distal end of the fluid delivery cannula and the fluid source for pumping fluid from the fluid source to the proximal end of the fluid delivery cannula.

10. The system of claim 1, wherein the fluid delivery cannula is a gravity fed fluid delivery cannula.

11. The system of claim 1, further comprising:
a valve fluidly coupled to the fluid delivery cannula, the valve configured to control fluid movement into the fluid delivery cannula or through at least a portion of the fluid delivery cannula.

12. The system of claim 11, wherein the valve is communicatively coupled to the controller such that the controller provides a signal to the valve to cause the valve to open or close.

13. The system of claim 1, further comprising:
a user interface communicatively coupled to the controller.

14. The system of claim 13, wherein the user interface is configured to provide an alert to a user when the fluid infiltration event is determined by the controller.

15. A fluid infiltration detection device, comprising:
an optical sensor optically coupled to a fluid delivery cannula delivering fluid from a fluid source therethrough and positioned at a location remote from a distal end of the fluid delivery cannula, the optical sensor configured to generate an electronic signal based on one or more characteristics of light sensed in the interior of the fluid delivery cannula when the fluid from the fluid source passes therethrough; and
a controller comprising a processor and a non-transitory, processor-readable storage medium, the non-transitory, processor-readable storage medium comprising programming instructions stored thereon that, when executed, cause the processor to:
receive the electronic signal from the optical sensor,
determine, based on the electronic signal, the one or more characteristics of the light,
determine, based on the one or more characteristics of the light, a fluid infiltration event at the distal end of the fluid delivery cannula, and
provide an alert indicating the fluid infiltration event.

16. The fluid infiltration detection device of claim 15, further comprising a light emitting device that emits light that is carried through an interior of the fluid delivery cannula, reflected at a target site adjacent to the distal end of the fluid delivery cannula, and sensed by the optical sensor.

17. The fluid infiltration detection device of claim 15, wherein the programming instructions that cause the processor to determine the fluid infiltration event further cause the processor to measure a brightness and/or intensity of the light at the distal end of the fluid delivery cannula.

18. A method for detecting fluid infiltration, the method comprising:
causing fluid to move to a distal end of a fluid delivery cannula to a target site;
sensing, via an optical sensor, light emitted from the target site and carried through at least a portion of the fluid delivery cannula,
measuring one or more characteristics of the light; and
determining a fluid infiltration event based on the one or more characteristics of the light.

19. The method of claim 18, further comprising:
measuring one or more baseline characteristics of the light prior to causing the fluid to move, wherein measuring the one or more characteristics of the light comprises measuring the one or more characteristics after causing the fluid to move.

20. The method of claim 19, further comprising:
comparing the one or more baseline characteristics to the one or more characteristics, wherein determining the fluid infiltration event is based on the comparing.
